(19) United States
(12) Patent Application Publication
(10) Pub. No.: US 2013/0245695 A1
(43) Pub. Date: Sep. 19, 2013

(54) VETERINARY PLATE

(71) Applicant: SYNTHES USA, LLC, West Chester, PA (US)

(72) Inventors: Jean BORDEAUX, West Chester, PA (US); Brian BEALE, Houston, TX (US); Randy J. BOUDRIEAU, N. Grafton, MA (US); Michael P. KOWALESKI, N. Grafton, MA (US); Rico VANNINI, Regensdorf (CH); Aldo VEZZONI, Cremona (IT)

(73) Assignee: SYNTHES USA, LLC, West Chester, PA (US)

(21) Appl. No.: 13/796,086

(22) Filed: Mar. 12, 2013

Related U.S. Application Data
(60) Provisional application No. 61/610,707, filed on Mar. 14, 2012.

(51) Int. Cl. A61B 17/80

(52) U.S. Cl. CPC A61B 17/8066
USPC 606/280

(57) ABSTRACT

A bone fixation device includes a plate body which includes (a) a first body portion lying in a first plane, (b) a second body portion lying in a second plane and (c) an interface body portion lying in a third plane. The first plane is angled relative to the second plane and the third plane is angled relative to the first and second planes. A longitudinal axis of the first body portion is angled with respect to a longitudinal axis of the second body portion. The first body portion has first and second openings extending therethrough. The second body portion has third and fourth openings extending therethrough.

### PRIORITY CLAIM

The present application claims priority to U.S. Provisional Appln. Ser. No. 61/610,707 entitled “Veterinary Plate” filed on Mar. 14, 2012, the entire disclosure of which is incorporated herein by reference.

### BACKGROUND INFORMATION

Triple Pelvic Osteotomy (TPO) is typically performed on immature canines to treat coxofemoral joint subluxation, changing the orientation of the acetabulum to allow improved congruity and coverage of the femoral head to increase coxofemoral joint stability. Increasing this stability of the joint helps to maintain normal joint mechanics and minimizes the subsequent development of osteoarthritis. The TPO procedure is performed by first performing an osteotomy of the pubis with a ventral approach to the hip. A second osteotomy of the ischium is then performed with a caudal approach directly caudal to the ischium, followed by a third osteotomy of the ilium with a craniolateral approach to the hip and ilial shaft. Once all three osteotomies have been made, the acetabulum is rotated to improve the congruity of the joint and ensure adequate capture the femoral head. A TPO plate is used to secure the rotation. The TPO procedure is subject to several potential complications, including sacral screw loosening, pelvic narrowing and excessive head coverage. In another procedure known as a Double Pelvic Osteotomy (DPO), only the pubic and ilial osteotomies are performed, without performing the ischial osteotomy, thus relying on the flexibility of the pubic symphysis and the ischiatic table. The DPO technique reduces issues of pelvic narrowing post-operation due to the continued integrity of the ischium, which maintains the pelvic width and prevents ventral displacement of the pelvic floor as seen with bilateral DPO. More importantly, maintaining ischial integrity increases the overall stability of the fixation, which results in better patient comfort and function early postoperatively. The DPO technique is more difficult to perform than the TPO because the acetabulum is significantly more difficult to rotate with the ischium intact. Furthermore, the DPO procedure also is subject to the additional complication of ilial screw pullout because of the elastic memory of the pelvis (and intact ischium) of the rotated acetabular segment. Regardless of the method utilized to rotate the acetabular segment, the surgeon must perform an accurate rotation and be able to secure this bone segment rigidly, which requires the appropriate implant.

### SUMMARY OF THE INVENTION

The present invention is directed to a bone fixation device comprising a plate body having a first body portion lying in a first plane, a second body portion lying in a second plane and an interface body portion lying in a third plane. The first plane is angled relative to the second plane an angle fixed relative to the first plane and the third plane is angled relative to the first and second planes. A longitudinal axis of the first body portion is angled with respect to a longitudinal axis of the second body portion. The first body portion has first and second openings extending therethrough and the second body portion has third and fourth openings extending therethrough.

### BRIEF DESCRIPTION OF THE DRAWINGS

FIG. 1 shows a first perspective view of a exemplary bone plate according to the present invention implanted in an ilial shaft portion;

FIG. 2 shows a second perspective view of the bone plate of FIG. 1;

FIG. 3 shows a third perspective view of the bone plate of FIG. 1;

FIG. 4 shows a first perspective view of a bone plate according to a first alternate embodiment of the invention;

FIG. 5 shows a second perspective view of the bone plate of FIG. 4;

FIG. 6 shows a first view of cranial screw trajectories of the bone plate of FIG. 1;

FIG. 7 shows a second view of cranial screw trajectories of the bone plate of FIG. 1;

FIG. 8 shows a first view of caudal screw trajectories of the bone plate of FIG. 1;

FIG. 9 shows a second view of caudal screw trajectories of the bone plate of FIG. 1;

FIG. 10 shows another view of the screw trajectories of the bone plate of FIG. 1

FIG. 11 shows a perspective view of a bone plate according to a second alternate embodiment of the invention;

FIG. 12 shows a perspective view of a bone plate according to a third alternate embodiment of the invention;

FIG. 13 shows a first perspective view of a bone plate according to a fourth alternate embodiment of the invention;

FIG. 14 shows a second perspective view of the bone plate of FIG. 13;

FIG. 15 shows a first perspective view of a bone plate according to a fifth alternate embodiment of the invention; and

FIG. 16 shows a second perspective view of the bone plate of FIG. 15.

### DETAILED DESCRIPTION

The present invention may be further understood with reference to the following description and the appended drawings, wherein like elements are referred to with the same reference numerals. The present invention relates generally to devices and methods for the stabilization of joints. Specifically, the present invention relates to methods and devices for changing an orientation of a shallow acetabulum to allow better coverage of the head of a femur of a cat or dog in connection with a TPO procedure. However, the exemplary bone plate according to the invention may also be used in conjunction with a DPO procedure. Specifically, the exemplary bone plate according to the invention may be used in either a DPO or a TPO procedure, with a surgeon selecting the procedure to conform to the requirements of the animal to be treated. The exemplary bone plate according to the invention has been designed to provide a stable and reliable fixation for both TPO and DPO procedures. The exemplary system and method according to the invention are configured increase a strength of the sacrum after implantation thereover by increasing a purchase of bone screws within the sacrum. Additionally, since the exemplary device according to the invention is configured for use with locking screws, the possibility of loosening of screws after implantation is minimized. As would be understood by those skilled in the art, the exemplary system and method disclosed herein may be employed in conjunction with bone fixation procedures for any animal by modifying the dimensions and shape of the apparatus to suit the particular anatomy. It is noted that directional terms assigned to components of the device of the present invention are labeled in accordance with a position of the device when in an operative configuration, as shown in FIG. 10 and described in greater detail below. However, those skilled in the art will understand that these terms are used to describe an exemplary procedure and are not intended to limit the invention. That is, the device may be oriented in other directions if so desired—e.g., when employed in different animals—as would be understood by those skilled in the art.

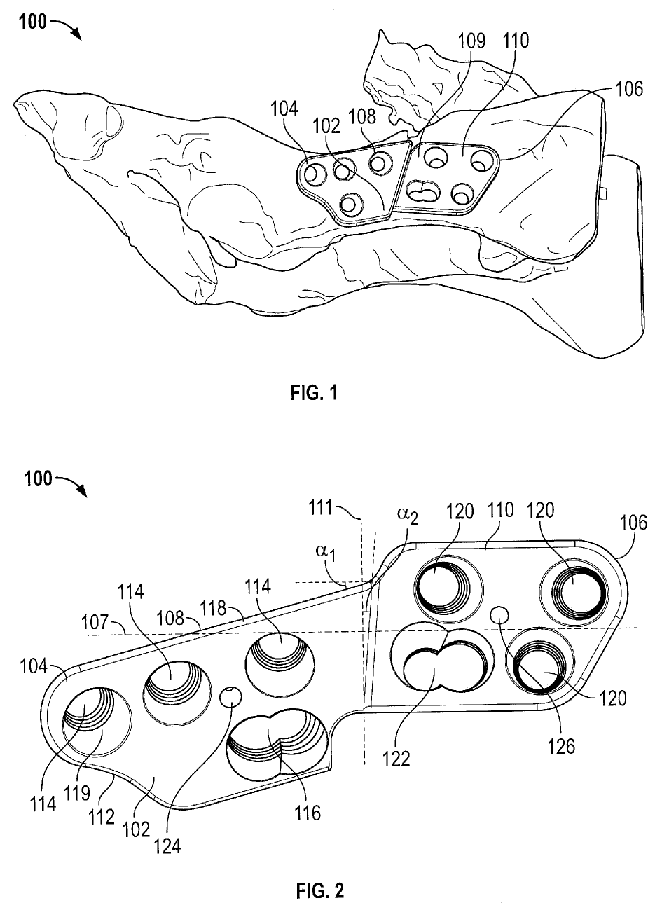

As shown in FIGS. 1-10, a device 100 according to an exemplary embodiment of the invention comprises a substantially elongated plate body 102 extending from a first end 104 to a second end 106 along a longitudinal axis 107. The plate body 102 comprises a first body portion 108 and a second body portion 110 axially offset from each other, as will be described in greater detail below. The plate body 102 is formed of a biocompatible material such as a metal or metal alloy (e.g., Stainless Steel, etc.) or a polymer (e.g., “PEEK”, etc.) having a rigidity selected to provide a desired degree of stability to the performed osteotomies when implanted, as those skilled in the art will understand. The first body portion 108 has a substantially rectangular cross-section with a caudal relief 112 formed adjacent the first end 104. The caudal relief 112 defines an arc-shaped cutout from the first body portion 108 configured to avoid interfering with the tuberosity of the rectus femoris muscle in an operative configuration, as those skilled in the art will understand. The first body portion 108 is angled at an angle of α₁ relative to the longitudinal axis 107 of the second body portion 110 of the plate body 102. In an exemplary embodiment, the angle α₁ is approximately 15°, although any other angular offset may be used as dictated by the anatomy without deviating from the scope of the invention. As those skilled in the art will understand, the angle α₁ is selected to provide a better fit of the first body portion 108 against the bone—i.e., to conform to a shape and dimension of the bone after it has been rotated to a corrected alignment in accordance with a DPO procedure. The first body portion 108 also comprises a plurality of bone fixation holes 114, 116 extending therethrough and configured to permit insertion of bone fixation screws 130 therethrough. The bone fixation holes 114, 116 may be threaded or unthreaded as those skilled in the art will understand. In one embodiment, the first body portion 108 comprises three bone fixation holes 114 disposed adjacent a dorsal wall 118 thereof. Each of the fixation holes 114 has a substantially conical shape and includes a chamfer relief 119 configured to seat a head of the bone fixation element 130 inserted therethrough. However, those skilled in the art will understand that any or all of the holes 114 may have any other desired configuration. Each of the fixation holes 114 extends through the first body portion 108 at an angle selected to ensure that a bone fixation element 130 inserted therethrough enters a target location relative of the bone, as will be described in greater detail with respect to FIGS. 6-10. Specifically, the fixation holes 114, 116 may be angled so that bone fixation screws 130 inserted therethrough advance into portions of bone having increased density, thereby increasing a holding strength of the plate body 102 with the bone, as those skilled in the art will understand. The fixation hole 116 of this embodiment is a combination hole configured to permit a physician to select a position and angle of the bone fixation element 130 inserted therethrough, as those skilled in the art will understand. It is noted that although the first body portion 108 is depicted with three fixation holes 114 and one combination hole 116, any number and combination of the above may be employed without deviating from the scope of the invention. Furthermore, the fixation holes 114, 116 may be positioned in any arrangement over the first body portion 108 without deviating from the scope of the invention.

The second body portion 110 extends substantially parallel to the longitudinal axis of the plate body 102 and comprises a plurality of bone fixation holes 120, 122 extending therethrough. Specifically, the second body portion 110 may comprise three fixation holes 120 and one combination hole 122. Each of the fixation holes 120 may extend through the second body portion 110 at a predetermined angle selected to permit the bone fixation element 130 inserted therethrough to be positioned at a desired location relative to the bone. Although the second body portion 110 is depicted with three fixation holes 120 and one combination hole 122, any number and combination of the above may be employed without deviating from the scope of the invention. Furthermore, the fixation holes 120, 122 may be positioned in any arrangement over the second body portion 110 without deviating from the scope of the invention. As those skilled in the art will understand, the use of combination fixation holes 116, 122 on each of the first and second body portions 108, 110 allows for compression of the bone regardless of whether a TPO or DPO technique is used. Specifically, by placing a combination hole in each of the first and second body portions 108, 110, a veterinarian or other user may secure either of the first or second body portions 108, 110 to the bone in a first step of a bone fixation procedure, as will be described in greater detail later on. The bone fixation holes 114, 116, 120, 122 according to the invention may be configured and dimensioned to receive locking or cortical screws (e.g, 3.5 mm screws, 2.7 mm. screws, etc.). In an exemplary embodiment, each of the first and second body portions 108, 110 comprises four fixation holes extending therethrough.

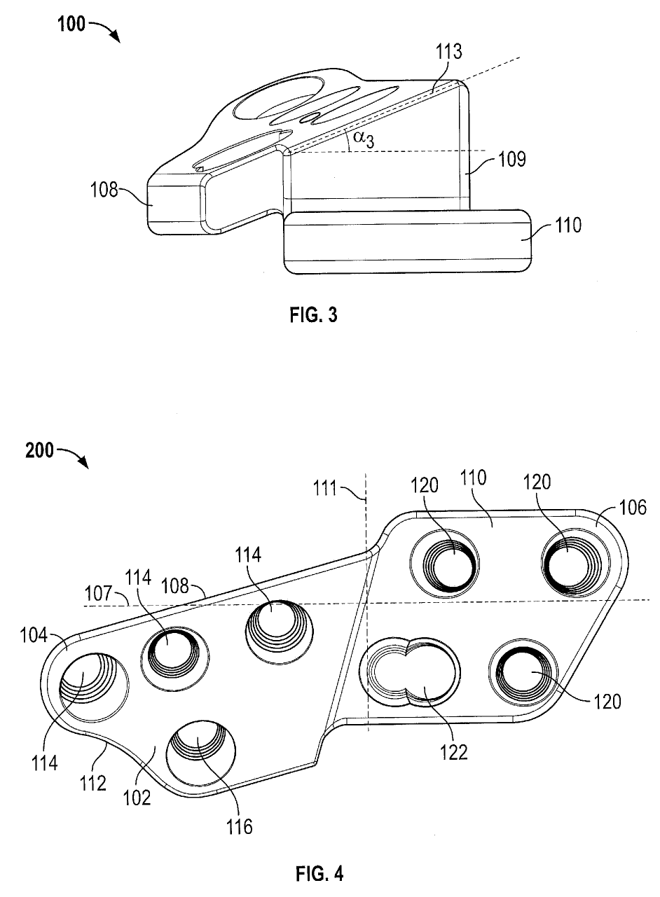

As shown in FIG. 3, the second body portion 110 resides in a plane axially offset from a plane housing the first body portion 108 by an angle α₃, as will be described in greater detail later on. Furthermore, the planes housing the first and second body portions 108, 110 extend along longitudinal axes angled relative to one another at an angle selected to conform to the anatomy of the bone. Specifically, the planes of the first and second body portions 108, 110 are oriented to permit the plate 102 to be seated substantially flush against the bone in an operative configuration.

The first body portion 108 is connected to the second body portion 110 by an interface portion 109 lying in a plane substantially perpendicular to the plane housing the second body portion 110. The interface portion 109 extends at an angle α₂ relative to an orthogonal axis 111 of the plate body 102. In an exemplary embodiment, the angle α₂ is approximately 5°. However, any other measurement for the angle α₂ may be used without deviating from the scope of the invention. In one example, the angle α₂ may be in the range of approximately 0-15°. In an exemplary embodiment, the angle α₃ corresponds to a desired angle of rotation required to stabilize a hip joint of the animal being treated. As shown in FIG. 3, a lateral wall 113 of the interface portion 109 is angled at an angle of α₃ relative to the plane housing the second body portion 110. In an exemplary embodiment, the angle α₃ may be one of approximately 20°, 25° and 30° although any angle in the range of 15-40° may be used without deviating from the scope of the invention.

The first and second body portions 108, 110 comprise Kirschner-wire (“K-wire”) holes 124, 126, respectively, positioned substantially centrally on the corresponding one of the first and second body portions 108, 110 to aid in placement thereof over the bone. Alternatively, the K-wire holes 124, 126 may be positioned anywhere else on the plate body 102 without deviating from the scope of the invention as long as each of the first and second body portions 108, 110 comprises one K-wire hole. In another embodiment, only one of the first and second body portions 108, 110 may be provided with a K-wire hole.

The plate body 102 according to the invention is configured and dimensioned to be placed over a target portion of bone. In one embodiment, a length of the plate body 102 along the longitudinal axis 107 is approximately 55 mm. It is noted, however, that any other length of the plate body 102 may be used without deviating from the scope of the invention. A thickness of the plate body 102 is approximately 4 mm and a height of the second body portion 110 is approximately 16 mm.

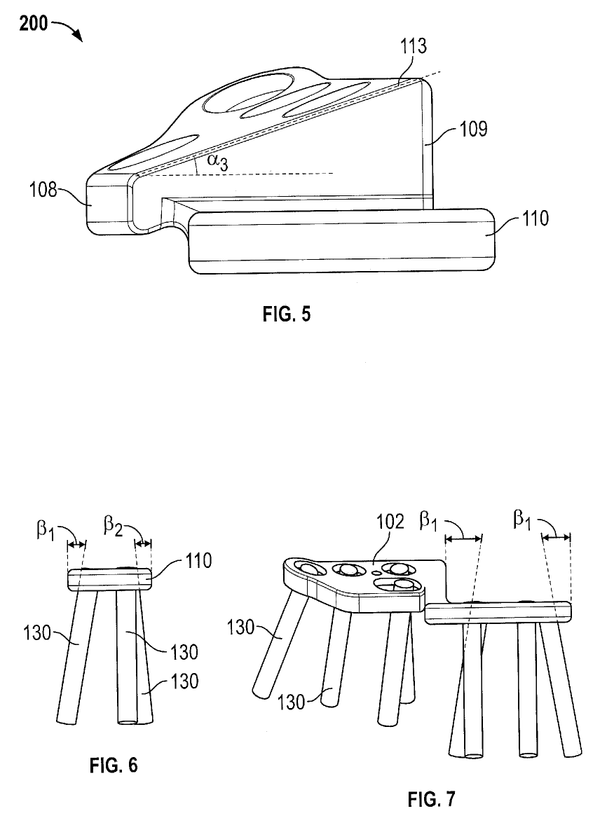

As shown in FIGS. 4-5, a device 200 according to an alternate embodiment of the invention, is substantially similar to the device 100, with like elements referenced with like reference numerals. An angle α₂ extends between the interface portion 109 and the orthogonal axis 111 of the plate body 102, the angle α₂ being in the range of approximately 0-15°, although any other angle may also be used without deviating from the scope of the invention. Additionally, the device 200 is formed with the angle α₃ between the lateral wall 113 of the interface portion 109 and the plane housing the second body portion 110 which may for example, be between 20° and 35°. More specifically, the angle α₃ may be approximately 20°, 25° or 30°. It is noted however that the angle α₃ may have any other magnitude without deviating from the scope of the invention. The height of the second body portion 110 of the device 200 in this embodiment is approximately 20 mm.

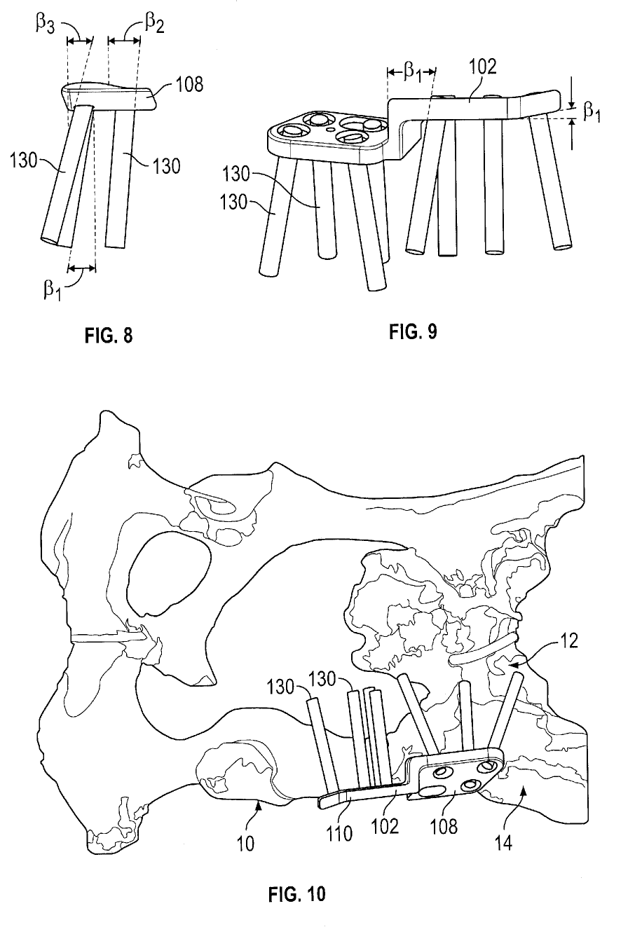

FIGS. 6-9 depict the exemplary trajectories of the bone fixation screws 130 inserted through the plate body 102, which trajectories are defined by the angles along which the bone fixation holes 114, 116, 120, 122 extend through the plate body 102. The bone fixation screws 130 may be locking screws, cancellous screws or cortex screws. In an exemplary embodiment, the bone fixation holes are angled so that adjacent pairs of bone fixation screws 130 diverge away from one another, as shown in FIG. 6. Specifically, a first one of the bone fixation screws 130 may be angled at an angle β₁ of approximately 10° relative to a perpendicular axis (not shown) intersecting the second body portion 110. A second one of the bone fixation screws may be angled at an angle β₂ of approximately 5° relative to the perpendicular axis (not shown), the angle β₂ extending away from the angle β₁ such that the first and second bone fixation screws diverge away from one another. A third bone fixation screw 130 may be angled at an angle β₃ of approximately 15° relative to a perpendicular axis (not shown) intersecting the second body portion 110, as shown in FIG. 8. As those skilled in the art will understand, the diverging angles provide the opportunity to reduce interference of the bone fixation screws 130 and instruments (e.g., drill guides) with soft tissue. However, in another embodiment (not shown), the bone fixation holes 114 may be substantially parallel to one another or angled so adjacent pairs of the bone fixation screws converge toward one another. As those skilled in the art will understand, this embodiment aids in capturing specific portions of the anatomy (e.g., strong portions of bone) and may also aid in reducing interference with soft tissue and instruments. In yet another embodiment, the plate body 102 may be configured with any combination of converging, diverging and parallel bone fixation holes 114, 116, 120, 122.

FIG. 10 depicts the device 100 in an operative configuration implanted over an ilial shaft 10 and ilial wing 14, as will be discussed in greater detail below.

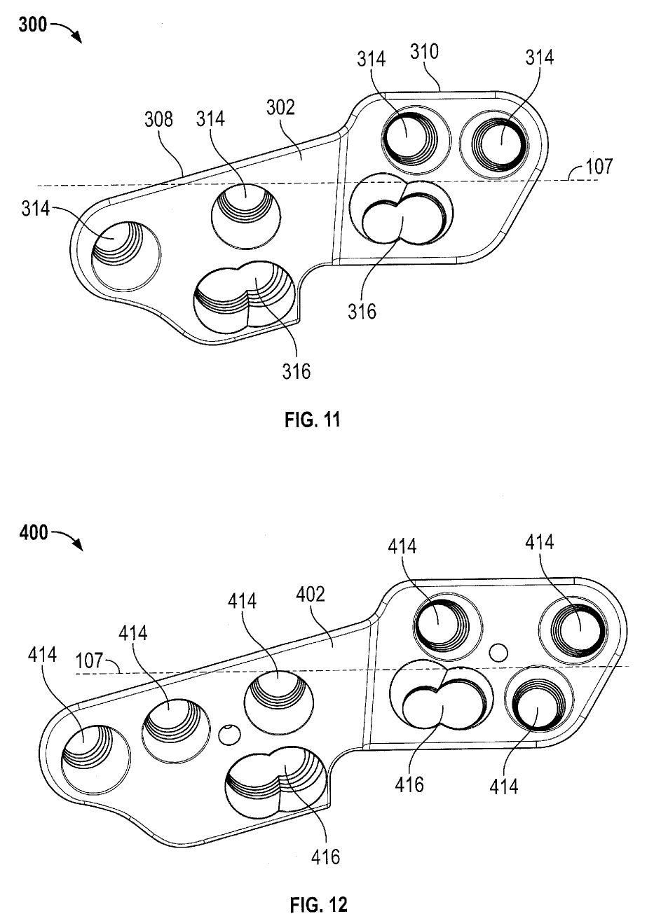

As shown in FIG. 11, a device 300 according to another embodiment of the invention is substantially similar to the device 100 of FIGS. 1-3 with a length of a plate body 302 along the longitudinal axis 107 being, for example, approximately 55 mm. However, while the first and second body portions 108, 110 each includes three standard bone fixation holes and one combination hole, each of the first and second portions 308, 310, respectively, of the device 300 includes only two fixation holes 314 and a single combination hole 316. This allows the device 300 to be made shorter than the device 100. For example, the device 300 may be made shorter than 55 mm permitting use in smaller animals, as those skilled in the art will understand.

As shown in FIG. 12, a device 400 according to yet another embodiment of the invention is substantially similar to the device 100 of FIGS. 1-3 except that fixation holes 414, 416 extending through the plate body 402 are smaller in diameter. For example, the holes 414, 416 may be configured and dimensioned to receive screws having a diameter of approximately 2.7 mm, permitting the device 400 to be made smaller than the device 100. This makes possible the production of a device 400 which is shorter than 55 mm permitting the use thereof in smaller animals, as those skilled in the art will understand.

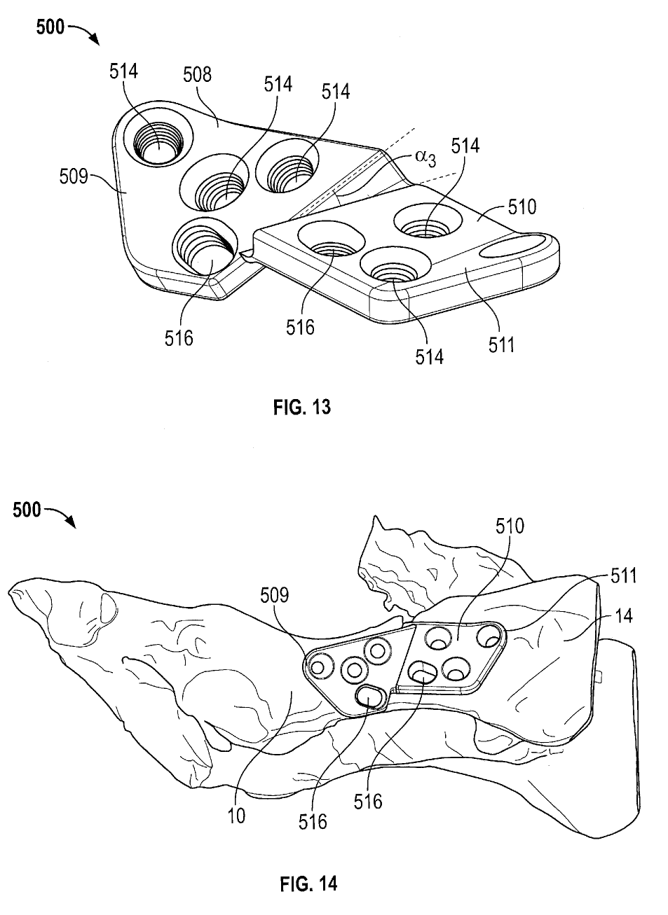

As shown in FIGS. 13-14, a device 500 according to another embodiment of the invention is substantially similar to the device 100 of FIGS. 1-3 with the exception of the placement of bone fixation holes 514, 516. Furthermore, a point of intersection of first and second body portions 508, 510 may be altered as necessary to conform to a particular anatomy of a target bone, as those skilled in the art will understand. It is therefore noted that the illustrations depicted herein are exemplary only and that any other point of intersection of the first and second body portions 508, 510 may be used without deviating from the scope of the invention. The first and second body portions 508, 510 may also comprise deflected wall portions 509, 511, respectively, configured to conform to the anatomy of the ilial shaft 10 when positioned thereover. Each of the wall portions 509, 511 deflects away from the plane housing the corresponding one of the first and second body portions 508, 510 by an angle selected to match the corresponding bone geometry. In one exemplary embodiment, the deflection may be approximately 10° relative to a plane housing the second body portion 510. It is noted that any of the devices disclosed herein may comprise any number and arrangement of deflected wall portions without deviating from the scope of the invention.

In accordance with an exemplary method according to the invention, two or three osteotomies are made in the bone in accordance with one of a TPO and a DPO procedure, wherein the surgeon may determine, prior to the surgery, which method is most suitable for the animal. Specifically, if the surgeon determines a TPO procedure is necessary, a first osteotomy is made in the pubic bone, a second osteotomy in the ischium and a third osteotomy in the ilium The osteotomies may be made via any known device, as those skilled in the art will understand. Once the osteotomies have been made, the surgeon rotates the acetabulum sufficiently to seat the head of the femur into a desired orientation within the acetabulum. The device 100 may then be positioned as shown over the ilial shaft 10 under guidance of K-wires inserted through the K-wire holes 124, 126. As those skilled in the art will understand, if a DPO procedure is used, only two osteotomies are performed, one in the pubis and the other in the ischium. The device 100 is positioned caudally over the osteotomy in the ilium, as those skilled in the art will understand. In an exemplary method, the first body portion 108, located caudally along the device 100, may then be secured to the bone first. However, those skilled in the art will understand that regardless of the orientation (e.g., right or left), in this example, a caudally located portion of the device 100 is secured to the bone first. Furthermore, as those skilled in the art will understand, the order of the steps of the surgical steps disclosed herein is exemplary only. Any other order of the steps may be employed to conform to the requirement of a particular application, surgeon preference, etc. without deviating from the scope of the invention.

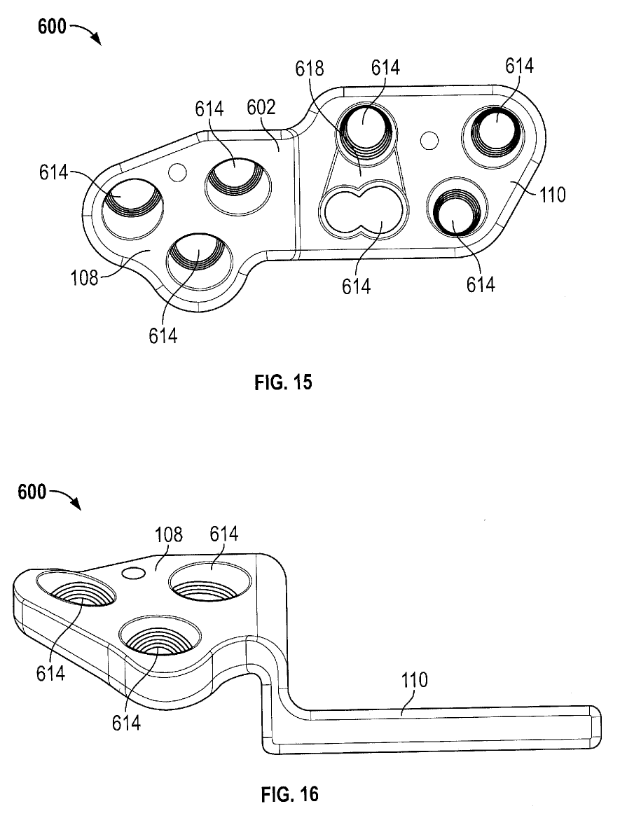

FIGS. 15-16 depict a device 600 having a plate body 602 in accordance with another embodiment of the invention, the device 600 being formed substantially similar to the device 100 of FIGS. 1-3 with the exception of the number and placement of bone fixation holes 614, 616. That is, the first body portion 108 of the device 600 includes three fixation holes 614 while the second body portion 110 includes three fixation holes 614 and one combination hole 616. The second body portion 110 further comprises a recess 618 extending between the combination hole 616 and an adjacent one of the fixation holes 614. As those skilled in the art will understand, the recess 618 serves to prevent the formation of sharp edges on an outer surface of the plate body 602 after performing a milling procedure therethrough. A nominal separation of 1 mm. is provided between the holes 614, 616. It is therefore noted that the illustrations depicted herein are exemplary only and that any other size, shape and position of the recess 618 may be used without deviating from the scope of the invention.

It will be apparent to those skilled in the art that various other modifications and variations may be made in the structure and the methodology of the present invention, without departing from the spirit or scope of the invention. Thus, it is intended that the present invention cover modifications and variations of the invention provided that they come within the scope of the appended claims and their equivalents.

What is claimed is:

1. A bone fixation device comprising:
a plate body comprising a first body portion lying in a first plane, a second body portion lying in a second plane and an interface body portion lying in a third plane, wherein:
the first plane is angled relative to the second plane and the third plane is angled relative to the first and second planes;
a longitudinal axis of the first body portion is angled with respect to a longitudinal axis of the second body portion;
the first body portion has first and second openings extending therethrough; and
the second body portion has third and fourth openings extending therethrough.

2. The bone fixation device of claim 1, wherein the first and third openings are combination holes.

3. The bone fixation device of claim 1, wherein the plate body is configured for use in one of a Triple Pelvic Osteotomy and a Double Pelvic Osteotomy.

4. The bone fixation device of claim 1, wherein a first end of the first body portion comprises a caudal cutout relief shaped and positioned so that, when implanted at a desired position on a bone, contact with a tuberosity of the rectus femoris muscle is minimized.

5. The bone fixation device of claim 1, wherein the angles of the first and second planes are configured to conform to the anatomy of a target bone for fixation.

6. The bone fixation device of claim 1, wherein the first and second openings are configured so that bone fixation screws inserted therethrough diverge away from one another.

7. The bone fixation device of claim 1, wherein the first and second openings are configured so that bone fixation screws inserted therethrough converge toward one another.

8. The bone fixation device of claim 1, wherein the first and second openings are configured so that bone fixation screws inserted therethrough extend parallel to one another.

9. The bone fixation device of claim 1, wherein the angle of the longitudinal axis of the first body portion with respect to the longitudinal axis of the second body portion is approximately 5°.

10. The bone fixation device of claim 1, wherein the angle of the first plane relative to the second plane is approximately 15-40°.

11. The bone fixation device of claim 1, further comprising a first deflected portion formed on the first portion, the first deflected portion extending along a fourth plane offset relative to the first plane and a second deflection portion formed on the second portion, the second deflected portion extending along a fifth plane offset relative to the second plane.

12. A method for stabilizing a hip joint of an animal, comprising:
forming a plurality of osteotomies in an ilial body of the animal, the osteotomies being formed in accordance with one of a Double Pelvic Osteotomy and a Triple Pelvic Osteotomy;
inserting a bone plate over the bone, the bone plate having a plate body comprising a first body portion lying in a first plane, a second body portion lying in a second plane and an interface body portion lying in a third plane, wherein: the first plane is angled relative to the second plane and the third plane is angled relative to the first and second planes, a longitudinal axis of the first body portion is angled with respect to a longitudinal axis of the second body portion; and
securing the bone plate to the bone by inserting bone fixation screws through first and second openings extending through the first body portion and through third and fourth openings extending through the second body portion.

13. The method of claim 12, wherein insertion axes of the first and second openings are selected to one of diverge and converge relative to one another.

14. A bone fixation device comprising:
a plate body comprising a first body portion lying in a first plane, a second body portion lying in a second plane and an interface body portion lying in a third plane, wherein:
the first plane is angled relative to the second plane;
a longitudinal axis of the first body portion is angled with respect to a longitudinal axis of the second body portion;
the first body portion has first and second openings extending therethrough; and
the second body portion has third and fourth openings extending therethrough

15. The device of claim 14, wherein the third plane is angled relative to the first and second planes.

16. The device of claim 14, wherein the third plane extend orthogonal to the second plane.

17. The device of claim 14, wherein the angle of the first plane relative to the second plane is approximately 15-40°.

18. The device of claim 14, wherein the third opening is a combination hole.

19. The device of claim 17, wherein the first opening is a combination hole.

20. The device of claim 15, further comprising a first deflected portion formed on the first portion, the first deflected portion extending along a fourth plane offset relative to the first plane and a second deflection portion formed on the second portion, the second deflected portion extending along a fifth plane offset relative to the second plane.
